(19) United States
(12) Patent Application Publication
(10) Pub. No.: US 2023/0056888 A1
(43) Pub. Date: Feb. 23, 2023

(54) COMPOSITIONS COMPRISING CYANIDIN-3-GALACTOSIDE FOR USE AS VASORELAXANT

(71) Applicant: Evonik Operations GmbH, Essen (DE)

(72) Inventors: Rosario LIZIO, Muenster (DE); Michael SCHWARM, Alzenau (DE); Albino CARRIZZO, Castello del Matese (IT); Carmine VECCHIONE, Villapiana Scalo (IT)

(73) Assignee: Evonik Operations GmbH, Essen (DE)

(21) Appl. No.: 17/787,254

(22) PCT Filed: Dec. 16, 2020
(86) PCT No.: PCT/EP2020/086436

§ 371 Date: Jun. 17, 2022

(30) Foreign Application Priority Data
Dec. 19, 2019 (EP) 19218263.2

(51) Int. Cl. A61K 31/7048; A61K 36/45; A61P 9/08

(52) U.S. Cl. CPC A61K 31/7048; A61K 36/45; A61P 9/08; A61K 2236/333; A61K 2236/51

(57) ABSTRACT

A composition may be used in vascular relaxation, wherein the composition may include one or more anthocyanins.

The present invention is related to a composition for use in vascular relaxation.

It is estimated that about 18 million people died from cardiovascular diseases (CVDs) in 2015 worldwide, and more people die every year from CVDs than from any other cause. Actually, due to the impossibility to act on non-modifiable cardiovascular risk factors such as age, gender, genetics and ethnicity, the pharmacological therapy remains the unique validated clinical approach able to fight CVDs incidence and progression however, leading to a dramatic increase in global spending (Mishra and Monica 2019). Thus, discovering new substances able to evoke cardiovascular protection, is imperative. Protective or preventive substances are also insofar of high interest, as arterial stiffness, a predictor for CVD, is not only reversible via diet and exercise, but also via dietary components like resveratrol (Oh, 2018).

During the last years, epidemiological studies have identified a relationship between diet and CVD, but there is still considerable scientific uncertainty about the relationship between specific dietary components and cardiovascular risk (Schmitt and Ferro 2013; Carrizzo et al. 2019). A promising dietary group for cardiovascular protection are polyphenols, especially flavonoids, as they are inversely associated with blood pressure and lower risk of hypertension (Godos, et al., 2019). In this regard, anthocyanins, natural pigments belonging to the flavonoid family, are widely distributed in the human diet such as beans, fruits, vegetables, and red wine (Khoo et al. 2017). Actually, it is well-accepted that these natural products present in fruits and plant-derived-foods are relevant because of their potential health-promoting effects, as suggested by the available experimental and epidemiological evidence (Wallace 2011a). For this reason, interest in the biochemistry and biological effects of anthocyanin compounds has increased substantially during the last decade. It has been reported that anthocyanins exert positive effects on human health by reducing inflammatory processes and counteracting oxidative stress (de Pascual-Teresa, Moreno, and Garcia-Viguera 2010), improving the blood lipid profile, inhibiting the growth of cancerous cells (Hou 2003) and owning anti-obesity effects (Tsuda et al. 2003). With regard to CVD, anthocyanins from blueberries or red wine showed an improvement in flow mediated dilation (FMD), and augmentation index in human, as well as NO-dependent vessel relaxation in mice (Andriambeloson, et al., 1998; Curtis, et al., 2019; Rodriguez-Mateos, et al., 2019). Despite all their beneficial properties, the possible direct action of anthocyanins on the vasculature, both at functional and molecular levels, remains completely unknown.

Anthocyanins are water-soluble vacuolar pigments that may appear red, purple or blue, depending on the surrounding pH-value. Anthocyanins belong to the class of flavonoids, which are synthesized via the phenylpropanoid pathway. They occur in all tissues of higher plants, mostly in flowers and fruits and are derived from anthocyanidins by addition of sugars. Anthocyanins are glycosides of flavylium salts. Each anthocyanin thus comprises three component parts: the hydroxylated core (the aglycone); the saccharide unit; and the counterion. Anthocyanins are naturally occurring pigments present in many flowers and fruit and individual anthocyanins are available commercially as the chloride salts, e.g. from Polyphenols Laboratories AS, Sandnes, Norway. The most frequently occurring anthocyanins in nature are the glycosides of cyanidin, delphinidin, malvidin, pelargonidin, peonidin and petunidin.

It is known that anthocyanins, especially resulting from fruit intake, have a wide range of biological activities, including antioxidant, anti-inflammatory, antimicrobial and anti-carcinogenic activities, improvement of vision, induction of apoptosis, and neuroprotective effects. Particularly suitable fruit sources for the anthocyanins are cherries, bilberries, blueberries, black currants, red currants, grapes, cranberries, strawberries, cowberries, elderberries, saskatoon berries and apples and vegetables such as red cabbage, black scented rice (especially the varieties Chakhao Poireiton and Chakhao Amubi), blue maize, winter barley, etc. (Benvenuti et al., 2004; Escalante-Aburto et al., 2016 and Diczhazi et al, 2014). Bilberries, in particular Vaccinium myrtillus, and black currants, in particular Ribes nigrum, are especially suitable.

Although their beneficial action, it has been reported that anthocyanins frequently interact with other phytochemicals, exhibiting synergistic biological effects making contributions from individual components difficult to decipher. In fact, the majority of intervention studies investigating anthocyanins have used foods containing several types of polyphenols. Only few studies have been performed using compounds (i.e. Medox®) containing purified anthocyanins isolated from bilberries. On this regard, it has been demonstrated that anthocyanin supplementation for 3-weeks reduces several NF-kB-regulated pro-inflammatory chemokines and immunoregulatory cytokines (Karlsen et al. 2007). Other studies showed an effect on HDL-C upregulation and LDL-C downregulation after 12-weeks of consumption (Qin et al. 2009). However, an interesting study did not find similar effects on blood lipids after 500 mg of anthocyanins (cyanidin 3-glucoside) for 12 weeks (Curtis et al. 2009), and hypothesized that different anthocyanins may possess different bioactivities.

Bilberries contain diverse anthocyanins, including delphinidin and cyanidin glycosides and include several closely related species of the genus Vaccinium, including Vaccinium myrtillus (bilberry), Vaccinium uliginosum (bog bilberry, bog blueberry, bog whortleberry, bog huckleberry, northern bilberry, ground hurts), Vaccinium caespitosum (dwarf bilberry), Vaccinium deliciosum (Cascade bilberry), Vaccinium membranaceum (mountain bilberry, black mountain huckleberry, black huckleberry, twin-leaved huckleberry), Vaccinium ovalifolium (oval-leafed blueberry, oval-leaved bilberry, mountain blueberry, high-bush blueberry).

Dry bilberry fruits of V. myrtillus contain up to 10% of catechin-type tannins, proanthocyanidins, and anthocyanins. The anthocyanins are mainly glucosides, galactosides, or arabinosides of delphinidin, cyanidin, and—to a lesser extent—malvidin, peonidin, and petunidin (cyanidin-3-O-glucoside (C3G), delphinidin-3-O-glucoside (D3G), malvidin-3-O-glucoside (M3G), peonidin-3-O-glucoside and petunidin-3-O-glucoside). Flavonols include quercetin- and kaempferol-glucosides. The fruits also contain other phenolic compounds (e.g., chlorogenic acid, caffeic acid, o-, m-, and p-coumaric acids, and ferulic acid), citric and malic acids, and volatile compounds.

Black currant fruits (R. nigrum) contain high levels of polyphenols, especially anthocyanins, phenolic acid derivatives (both hydroxybenzoic and hydroxycinnamic acids), flavonols (glycosides of myricetin, quercetin, kaempferol, and isorhamnetin), and proanthocyanidins (between 120 and 166 mg/100 g fresh berries). The main anthocyanins are delphinidin-3-O-rutinoside (D3R) and cyanidin-3-O-rutinoside (C3R), but D3G and C3G are also found (Gafner, Bilberry—Laboratory Guidance Document 2015, Botanical Adulterants Program).

EP 1443948 A1 relates to a process for preparing a nutritional supplement (nutraceutical) comprising a mixture of anthocyanins from an extract of black currants and bilberries. Anthocyanins were extracted from cakes of fruit skin produced as the waste product in fruit juice pressing from V. myrtillus and R. nigrum. It could be shown that the beneficial effects of individual anthocyanins are enhanced if instead of an individual anthocyanin, a combination of different anthocyanins is administered orally, in particular a combination comprising both mono and disaccharide anthocyanins. It is thought that the synergistic effect arises at least in part from the different solubilities and different uptake profiles of the different anthocyanins.

In the context it was surprisingly found that extracts of black currants and bilberries exerts an important vasorelaxant effect of mice resistance arteries. This action is mediated by nitric oxide release through the intracellular signaling PI3K→Akt. Moreover, behind its capability to modulate vascular tone, it exerts also an important antioxidant effect though the modulation of NADPH oxidase enzyme. Interestingly, its cardiovascular properties are mediated by the selective action of different anthocyanins. Finally, the exposure of human dysfunctional vessels to berry extracts significantly reduces oxidative stress and improves NO bio-availability.

The experimental data with a dose-dependent administration of Healthberry 865® evokes specific endothelial vasorelaxation both in conduit and resistance arteries such as aorta, carotid and in femoral and mesenteric arteries, respectively. Its action is endothelium specific, since the mechanical removal of endothelial layer completely abrogate the vasoactive action of the compound. At this point, based on the effect evoked by Healthberry 865® on resistance arteries, and considering that the alteration of endothelium-dependent vasodilation in resistance arteries, but not in the brachial conduit artery has been associated with 5-year risk of a composite end-point of death, myocardial infarction, or stroke independently of major cardiovascular disease risk factors (Lind et al. 2011), the studies were focused on mice mesenteric arteries which better reproduce the function of human resistance arteries. The evaluation of Healthberry 865® evoked endothelial vasorelaxation in presence of a selective eNOS inhibitor completely abolished its vasorelaxant properties, thus leading to investigate possible mechanisms involved in eNOS activation. Different molecular mechanisms are involved in eNOS regulation, between these AMPK, PI3K, Akt represent the well known intracellular signaling able to modulate NO production (Li et al. 2018). Interestingly, the inhibition of AMPK by Dorsomorphin did not exert any effect on the vasorelaxant properties of Healthberry 865®, in contrast to that observed in presence of PI3K and Akt inhibitors, which are able to block its endothelial dependent vasorelaxation. Of note, the assessment of nitric oxide production by DAF-FM clearly revealed the capability of Healthberry 865® to induce nitric oxide production from endothelial layer, reproducing the action evoked by ACh, the gold standard molecule used to asses endothelial derived nitric oxide production. The analysis at molecular levels showed that in presence of PI3K or Akt inhibitors there is a significant reduction of eNOS activation, evaluated by the analysis of the phosphorylation site serine 1177, the main activation site of the enzyme.

The present invention is related to a composition for use in vascular relaxation,

wherein the composition comprises cyanidin 3-galactoside (C3Gal) according to the following formula:

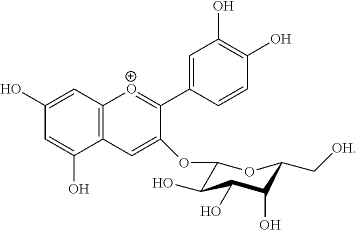

C3Gal, also known as ideain, is an anthocyanin found in black currants, bilberries and other fruits and can be used from a natural origin or can be synthesized in vitro or in vivo. C3Gal is the main anthocyanin in red-skinned or red-fleshed (for example Weirouge) apple varieties. It is also found in Chinese hawthorn fruits (Crataegus spp.). C3Gal is one of the anthocyanins present in bilberries (Vaccinium myrtillus) and cranberries (Vaccinium macrocarpon) and is the main anthocyanin in lingonberries (Vaccinium vitis-idaea). In the leaves of Quintinia serrata, the tawheowheo, a species of evergreen trees endemic to New Zealand, different patterns of anthocyanins (including C3Gal) are present to protect the shade-adapted chloroplasts from direct sun light. It is preferred to use extracts of fruits or cereals as a source of C3Gal, selected from bilberries, cranberries, cowberries, lingonberries, red, yellow and green apple, aronia, black chokeberry, black scented rice (Chakhao Poireton, Chakhao Amubi) and winter barley.

The composition further comprises one or more of the following anthocyanins: cyanidin-3-rutinoside, delphinidin-3-arabinoside and delphinidin-3-sambubioside.

In a preferred configuration, the composition comprises cyanidin 3-galactoside and cyanidin-3-rutinoside.

In another preferred configuration, the composition comprises cyanidin 3-galactoside and cyanidin-3-rutinoside and delphinidin-3-arabinoside.

In another preferred configuration, the composition comprises cyanidin 3-galactoside and delphinidin-3-arabinoside.

In another preferred configuration, the composition comprises delphinidin-3-sambubioside and cyanidin 3-galactoside.

In another preferred configuration, the composition comprises delphinidin-3-sambubioside and delphinidin-3-arabinoside.

In another preferred configuration, the composition comprises delphinidin-3-sambubioside and cyanidin-3-rutinoside and delphinidin-3-arabinoside.

For those embodiments, it is preferred, when the anthocyanins are present in the composition at a concentration of at least 5 μg/ml, preferably at least 10 μg/ml, more preferably at least 25 μg/ml, most preferably at least 50 μg/ml.

Information on anthocyanin content on different fruits can be found in the literature, such as for black currant red currant, black chokeberry, bilberry, cowberry, elderberry, (Benvenuti et al., 2004; Kahkonen et al., 2003; Wu et al., 2004), strawberry, sweet cherry and sour cherry (Jakobek et al., 2007), wild blueberries and Saskatoon berries (Hosseinian et al., 2007), rhubarb petioles (Takeoka et al., 2013), black scented rice Chakhao Poireton, Chakhao Amubi (Asem et al., 2015).

High amounts of delphinidin-3-arabinoside are present in bilberries and black scented rice.

High amounts of cyanidin-3-rutinoside are present in blackberry, black currant, red currant, sweet cherry, sour cherry, rhubarb, Saskatoon berry.

Therefore, a preferred combination of fruits/cereals comprises fruits or extracts of: black chokeberries, cowberries/lingonberries and bilberries or black scented rice and sweet cherry or black currant or red currant. In a further preferred embodiment comprises fruits or extracts from black currant, (black) chokeberries, bilberries, sweet cherry and cowberries/lingonberries or hibiscus or elderberries.

It is particularly preferred to provide mixtures with similar amounts of the beneficial anthocyanins, to ensure maximum vasorelaxant effects. Therefore, in an advantageous configuration of the present invention, the mixture comprises black currant, (black) chokeberry, bilberries and sweet cherry. The composition comprises anthocyanins and the anthocyanins are present in the composition at a concentration of at least 5 μg/ml, preferably at least 10 μg/ml, more preferably at least 25 μg/ml, most preferably at least 50 μg/ml.

In a preferred embodiment, the mixture comprises the specific fruits in defined ratios (in weight-%): black currant:(black) chokeberry:bilberries:sweet cherry in ratios of 0.5-5:0.25-2.5:15-35:50-70, more preferably 1:0.4:25:60.

In an alternative embodiment, the composition further comprises delphinidin-3-O-sambubioside and/or cyanidin-3-O-sambubioside, preferably form Hibiscus or an extract of Hibiscus (Ojeda et al., 2009) and/or from Elderberries or extract of Elderberries (M. Ciocoiu et al., 2009).

In one embodiment, the composition comprises an extract of black currants and bilberries.

In a preferred embodiment, the black currants are the fruit of Ribes nigrum and/or the bilberries are the fruit of Vaccinium myrtillus. It is further preferred, when the composition contains an extract from black currants and bilberries in a weight ratio of 0.5:1 to 1:0.5. In an advantageous configuration of the present invention, the composition is an extract of the pomaces from black currants and bilberries.

It is particularly preferred, when the composition comprises anthocyanins and the anthocyanins are present in the composition at a concentration of at least 25 weight-%, preferably at least 30 weight-%, or at least 35 weight-%, or at least 40 weight-%, or at least 45 weight-%, or at least 50 weight-%.

It is preferred, according to the present invention, when the extract is an alcoholic extract, preferably a methanol extract. The extract is preferably produced by a process comprising the steps of
extraction of black currants and/or bilberries,
purification via chromatography,
mixing of the extract(s) with water and
spray-drying of the mixture.

One example of such a process is disclosed in EP1443948.

In a preferred embodiment, the composition is for preventing or treating a disease or disorder selected from cardiovascular diseases, preferably atherosclerosis, hypertension, stroke, diabetes-related cardiovascular disfunctions, ischemia/reperfusion injury, hypercholesterolemia, coronary artery disease or chronic obstructive pulmonary disease (COPD).

The composition according to the present invention preferably contains at least three monosaccharide anthocyanins. Moreover, it preferably contains at least one monosaccharide anthocyanin in which the saccharide is arabinose or at least one disaccharide anthocyanin in which the disaccharide is rutinose. The composition preferably contains anthocyanins with at least two different aglycones, more preferably at least four. Especially preferably the composition contains anthocyanins in which the aglycone units are cyanidin, peonidin, delphinidin, petunidin, malvidin and optionally also pelargonidin. In one preferred embodiment, the composition also contains at least one trisaccharide anthocyanin. The disaccharide anthocyanins are more water-soluble than the monosaccharides; moreover, cyanidin and delphinidin anthocyanins are amongst the most water-soluble anthocyanins.

The anthocyanins can be from natural sources or from synthetic productions. Natural sources are preferably selected from fruits, flowers, leaves, stems and roots, preferably violet petal, seed coat of black soybean. Preferably anthocyanins are extracted from fruits selected from: acai, black currant, aronia, eggplant, blood orange, marion blackberry, black raspberry, raspberry, wild blueberry, cherry, queen Garnet plum, red currant, purple corn (Z. mays L.), concord grape, norton grape, muscadine grape, red cabbage, okinawan sweet potato, Ube, black rice, red onion, black carrot. Particularly suitable fruit sources for the anthocyanins are cherries, bilberries, blueberries, black currants, red currants, grapes, cranberries, strawberries, black chokeberry, and apples and vegetables such as red cabbage. Bilberries, in particular Vaccinium myrtillus, and black currants, in particular Ribes nigrum, are especially suitable. It is further preferred to use plants enriched with one or more of anthocyanins as natural sources, preferably plants enriched with delphinidin rutinoside.

The counterion in the anthocyanins in the composition of the invention may be any physiologically tolerable counter anions, e.g. chloride, succinate, fumarate, malate, maleate, citrate, ascorbate, aspartate, glutamate, etc. Preferably however the counterion is a fruit acid anion, in particular citrate, as this results in the products having a particularly pleasant taste. Besides the anthocyanins, the composition may desirably contain further beneficial or inactive ingredients, such as vitamins (preferably vitamin C), flavones, isoflavones, anticoagulants (e.g. maltodextrin, silica, etc.), desiccants, etc. The present invention is also related to a composition comprising extracts or fruits of black currant, (black) chokeberries, bilberries and sweet cherry, wherein the composition comprises anthocyanins and the anthocyanins are present in the composition at a concentration of at least 5 μg/ml, preferably at least 10 μg/ml, more preferably at least 25 μg/ml, most preferably at least 50 μg/ml.

It is preferred, when the composition further comprises extracts or fruits of cowberries or lingonberries or hibiscus or elderberries.

It is preferred, when the composition comprises black currant, (black) chokeberries, bilberries and sweet cherry in a ratio of 0.5-5:0.25-2.5:15-35:50-70. In another preferred embodiment, the composition comprises black currant, (black) chokeberries, bilberries and sweet cherry in a ratio of around 1:0.4:25:60.

### WORKING EXAMPLES

The berry extracts composition (Healthberry® 865; Evonik Nutrition & Care GmbH, Darmstadt, Germany) used in the present study is a dietary supplement consisting of 17 purified anthocyanins (all glycosides of cyanidin, peonidin, delphinidin, petunidin, and malvidin) isolated from black currant (Ribes nigrum) and bilberries (Vaccinium myrtillus).

The relative content of each anthocyanin in the Healthberry® 865 product was as follows: 33.0% of 3-O-b-rutinoside, 3-O-b-glucosides, 3-O-b-galactosides, and 3-O-b-arabinosides of cyanidin; 58.0% of 3-O-b-rutinoside, 3-O-b-glucosides, 3-O-b-galactosides, and 3-O-b-arabinosides of delphinidin; 2.5% of 3-O-b-glucosides, 3-O-b-galactosides, and 3-O-b-arabinosides of petunidin; 2.5% of 3-O-b-glucosides, 3-O-b-galactosides, and 3-O-b-arabinosides of peonidin; 3.0% of 3-O-b-glucosides, 3-O-b-galactosides, and 3-O-b-arabinosides of malvidin.

The 3-O-b-glucosides of cyanidin and delphinidin constituted at least 40-50% of the total anthocyanins.

The major anthocyanins contained in the berry extract used are cyanidin-3-glucoside, cyanidin-3-rutinoside, delphinidin-3-glucoside, delphinidin-3-rutinoside, cyanidin-3-galactoside and delphinidin-3-galactoside.

In addition to the anthocyanins mentioned above, the product also contained maltodextrin (around 40 weight-% of the composition), and citric acid (to maintain stability of anthocyanins). The amount of anthocyanin citrate is at least 25 weight-% of the composition. The composition is prepared from black currants and bilberries by a process comprising the steps of alcoholic extraction of black currants and bilberries, purification via chromatography, mixing of the extracts with maltodextrin citrate and water and spray-drying of the mixture. The product composition contains extracts of black currants and bilberries mixed in a weight ratio of around 1:1.

Materials:

Healthberry 865® (HB) was obtained from Evonik Nutrition & Care GmbH, Darmstadt (Germany) and single anthocyanins, Delfinidin-3-rutinoside (D3-rut), Cyanidin-3-rutinoside (C3-rut), Delphinidin-3-glucoside (DP3-glu), Cyanidin-3-glucoside (C3-glu), Petunidin-3-glucoside (PT3-glu), Delphinidin-3-galactoside (DP3-gal), Peonidin-3-galactoside (PEO3-gal), Delphinidin-3-arabinoside (DP3-ara), Malvidin-3-galactoside (MAL3-gal), Malvidin-3-glucoside (MAL3-glu), Cyanidin-3-galactoside (C3-gal), Cyanidin-3-arabinopyranoside (C3-arapy) and Delphinidin-3-sambubioside (DP3-samb) were obtained from Polyphenols AS, Sandnes (Norway). Primary antibodies and horseradish peroxidase (HRP)-labeled anti-rabbit or anti-mouse fragment immunoglobulin, and enhanced chemiluminescence for Western blotting detection reagent were purchased from Amersham Biosciences. All the inhibitors, powders and solvents necessary for the preparation of the buffers were purchased by Sigma-Aldrich.

Experimental Animals

All experiments involving animals were conformed to the Guide for the Care and Use of Laboratory Animals published by the US National Institutes of Health (NIH Publication No. 85-23, revised 2011) and were approved by review board. Wild-type C57BL/6 mice (weighing ˜25 g) (Jackson Laboratories, Bar Harbor, Me., USA) have been used to perform vascular reactivity and molecular studies.

Vascular Reactivity Studies

Aorta, carotid, femoral arteries and second-order branches of the mesenteric arterial tree were removed from mice to perform vascular studies. Vessels were placed in a wire or pressure myograph system filled with Krebs solution maintained at pH 7.4 at 37° C. in oxygenated (95% O₂/5% CO₂). First, an analysis of vascular reactivity curves was performed. In particular, vasoconstriction was assessed with 80 mmol/L of KCl or with increasing doses of phenylephrine (from 10-9 M to 10-6 M) in control conditions. Endothelium-dependent and -independent relaxations were assessed by measuring the dilatory responses of mesenteric arteries to cumulative concentrations of acetylcholine (from 10-9 M to 10-6 M) or nitroglycerine (from 10-9 M to 10-6 M) respectively, in vessels precontracted with phenylephrine at the dose necessary to obtain a similar level of precontraction in each ring (80% of initial KCl-evoked contraction). Caution was taken to avoid endothelial damage; functional integrity was reflected by the response to acetylcholine (from 10-9 M to 10-6 M).

Vascular responses were then tested administering increasing doses of Healthberry 865®-865 or single anthocyanins. Some experiments were performed in presence of selective inhibitors, such as phosphatidylinositol-4,5-bisphosphate 3-kinase inhibitor (LY274002, 10 μM, 1 h), Akt inhibitor (Akt inh, 1 μM, 1 h) or the NOS inhibitor N-ω-nitro-1-arginine methyl ester (L-NAME, 300 μM, 30 min) before data for dose-response curves were obtained.

Evaluation of NO Production by DAF

Production of NO was assessed as previously described (Carrizzo et al. 2016). Healthberry 865®-865 (100 μg/mL) or acetylcholine (10-6 M) was administered to the mesenteric artery in the last 30 min of 4-amino-5-methylamino-2,7,-difluorofluorescein diacetate (DAF-FM) incubation, alone and after 20 min exposure to L-NAME (300 μmol/L, 30 min). Mesenteric segments were cut in 5-μm thick sections, observed under a fluorescence microscope, subsequently counterstained with haematoxylin and eosin and observed under a light microscope.

Analysis of Total ROS Production

Dihydroethidium (DHE, Life Technologies) was used to evaluate production of reactive oxygen species (ROS) in mouse mesenteric arteries, as previously described. Briefly, vessels were incubated with 5 μM of DHE for 20 min and subsequently observed under a fluorescence microscope (Zeiss). Images were acquired by a digital camera system (Olympus Soft Imaging Solutions). A second estimation of total ROS production in mouse vessels was performed with the membrane-permeable fluorescent probe an analog of 2,7-Dichlorodihydrofluorescein (DCDHF), Dihydrorhodamine 123 (DHR123) (Invitrogen). After treatment, vessels were incubated with Krebs solution containing 5 μM DHR123 for 30 min at 37° C., and then washed two times with PBS prior to fluorescence measurement using a fluorescence microplate reader (TECAN infinite 200 Pro).

Evaluation of NADPH-Mediated O2—Production

To determine NADPH oxidase-mediated superoxide radical (O2-) production, we used the lucigenin-enhanced chemiluminescence assay, as previously described (Schiattarella et al. 2018). Vessels were homogenized in a buffer containing protease inhibitors (mmol/L: 20 monobasic potassium phosphate, 1 EGTA, 0.01 aprotinin, 0.01 leupeptin, 0.01 pepstatin, 0.5 phenylmethylsulfonyl fluoride, pH 7.0). Protein content was measured in an aliquot of the homogenate by Bradford method. In some experiments, cells and vessels were pre-incubated with pharmacological inhibitors before measurements. The reaction was started by the addition of NADPH (0.1 mmol/l) and lucigenin (5 μmol/l) to each well. The chemiluminescence was measured using Tecan Infinite Pro M200 multimode microplate at 37° C.

Gel Electrophoresis and Immunoblotting.

After isolation, arteries were solubilized in lysis buffer containing 20 mmol/L Tris-HCl, 150 mmol/L NaCl, 20 mmol/L NaF, 2 mmol/L sodium orthovanadate, 1% Nonidet, 100 μg/ml leupeptin, 100 μg/ml aprotinin and 1 mmol/L phenylmethylsulfonyl fluoride. Samples were left on ice for 30 minutes, centrifuged at 13000 g for 15 minutes and supernatants were used to perform Western immunoblot analysis. Total protein levels were determined using the Bradford method. 30 μg proteins were resolved on 8% SDS-PAGE, transferred to a nitrocellulose membrane and immunoblotted with anti-phospho-eNOS Serine 1177 (Cell Signaling, rabbit polyclonal antibody 1:800) and with anti-total-eNOS (Cell Signaling, mouse mAb 1:1000). HRP-conjugated secondary antibodies were used at 1:3000 dilution (Bio-Rad Laboratories). Protein bands were detected by ECL Prime (Amersham Biosciences) and quantitated with ImageJ software.

Statistical Analysis

Data are presented as mean±SEM. Statistical analysis was performed by 2-way ANOVA followed by Bonferroni post hoc test. Repeated measurements were analysed by One-way ANOVA followed Bonferroni post-hoc test. Differences were considered to be statistically significant at p<0.05.

### Example 1: Berry Extracts Evoke a Direct Vasorelaxant Action of Conduit and Resistance Arteries

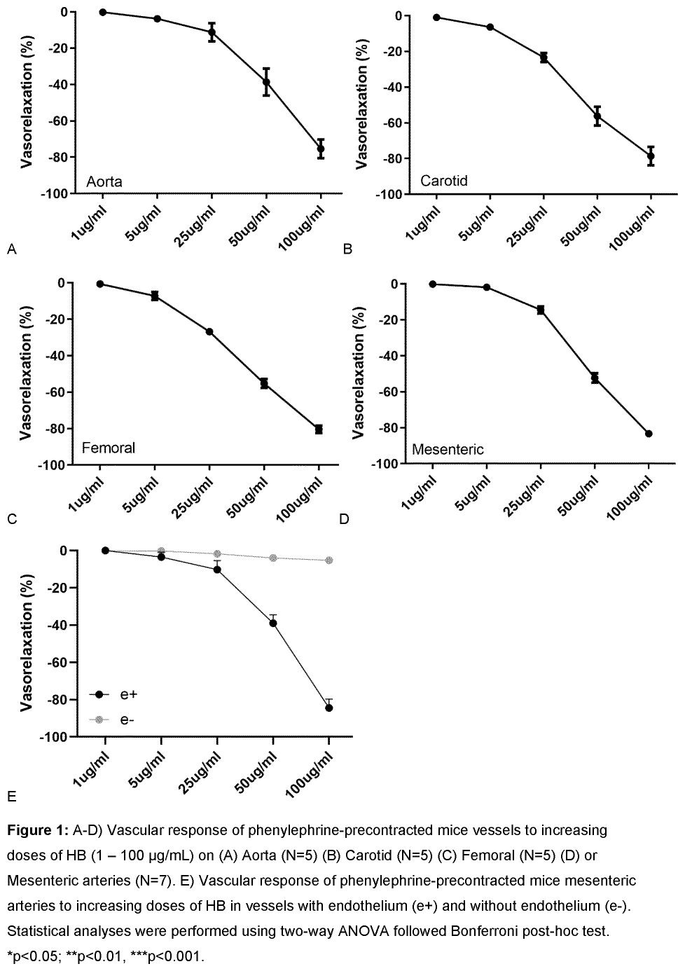

In order to evaluate the vascular properties of HB a first series of experiments were performed on different vascular districts, aorta and carotid arteries and femoral and mesenteric arteries, which represent respectively the prototypes of conduit and resistance arteries. Interestingly, the administration of increasing doses of HB (1 μg/mL to 100 μg/mL) is able to exert, per se, a direct vasorelaxant action on both kinds of vascular districts. As showed in FIG. 1, HB evokes a dose-dependent vascular effect, and at the maximal dose used of 100 μg/mL, was able to reach 80-90% of vasorelaxation in all studied vessels.

FIG. 1A-D) shows vascular response of phenylephrine-precontracted mice vessels to increasing doses of HB (1-100 μg/mL) on (A) Aorta (N=5) (B) Carotid (N=5) (C) Femoral (N=5) (D) or Mesenteric arteries (N=7). E) Vascular response of phenylephrine-precontracted mice mesenteric arteries to increasing doses of HB in vessels with endothelium (e+) and without endothelium (e−). Statistical analyses were performed using two-way ANOVA followed Bonferroni post-hoc test. *p<0.05; **p<0.01, ***p<0.001.

Based on the well-validated concept that alteration of resistance arteries exerts an important role in the development, and may contribute to the complications of cardiovascular disease, mice mesenteric arteries were characterized, which are considered the prototype of resistance vessels.

### Example 2: The Vascular Effect is Endothelial Nitric Oxide Synthase-Mediated

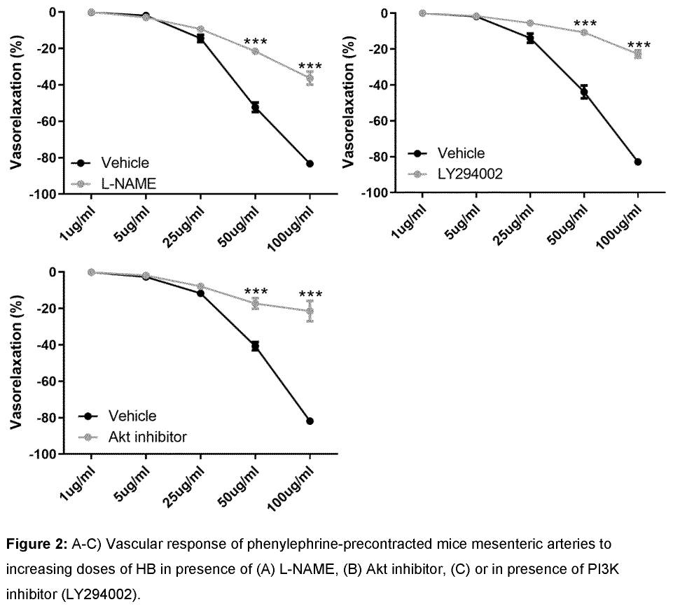

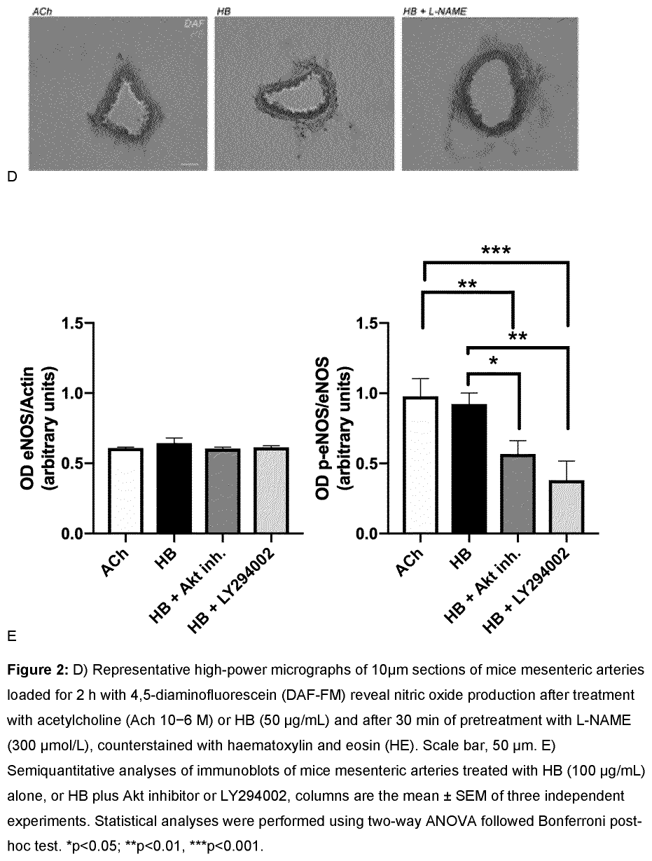

To investigate the possible vascular molecular mechanisms recruited by HB, further experiments on mice mesenteric arteries were performed, which is considered the prototype of resistance vessels involved in the blood pressure regulation. As showed in FIG. 2, the administration of increasing doses of HB to phenylephrine-preconstricted mesenteric arteries pre-treat with L-NAME, a well-validated endothelial Nitric Oxide Synthase (eNOS) inhibitor, showed a complete abolition of its vasorelaxant effect, thus suggesting that eNOS enzyme represents the endothelial target molecule necessary to translate the vascular effect of HB. To dissect the molecular mechanism involved, one of the most important modulators of eNOS metabolism, Akt, a serine/threonine-specific protein kinase that plays a key role in endothelium-dependent relaxation through eNOS enzyme were interrogated. Interestingly, the use of Akt inhibitor was able to completely block the vasorelaxant effect of HB. Generally, to activate Akt some upstream molecules able to elicit the activation of Nitric Oxide production are needed. In this regard, Phosphatidylinositol 3-Kinase (PI3K) represents one of the best characterized molecules involved in the intracellular activation of eNOS. In this experimental setting, the pre-treatment with LY294002, a strong chemical inhibitor of PI3K was able to abolish the vascular action of HB, confirming that PI3K/AKT/eNOS-dependent signalling pathway represents the molecular mechanism that transduces the vascular action of HB. The western blot analyses showed that HB is able, through PI3K and Akt, to positively modulate the Serine 1177 phosphorylation site of eNOS, the most important activation site of the enzyme that promotes NO production. Moreover, DAF-FM fluorescence induced by HB was comparable to that obtained with a classical agonist that evokes NO release, such as acetylcholine, and L-NAME pretreatment clearly abolishes endothelial nitric oxide release (FIG. 2).

FIG. 2A-C) show vascular response of phenylephrine-precontracted mice mesenteric arteries to increasing doses of HB in presence of (A) L-NAME, (B) Akt inhibitor, (C) or in presence of PI3K inhibitor (LY294002).

FIG. 2 D) show representative high-power micrographs of 10 μm sections of mice mesenteric arteries loaded for 2 h with 4,5-diaminofluorescein (DAF-FM) reveal nitric oxide production after treatment with acetylcholine (Ach 10-6 M) or HB (50 μg/mL) and after 30 min of pretreatment with L-NAME (300 μmol/L), counterstained with haematoxylin and eosin (HE). Scale bar, 50 μm. E) Semiquantitative analyses of immunoblots of mice mesenteric arteries treated with HB (100 μg/mL) alone, or HB plus Akt inhibitor or LY294002, columns are the mean±SEM of three independent experiments. Statistical analyses were performed using two-way ANOVA followed Bonferroni post-hoc test. *p<0.05; **p<0.01, ***p<0.001.

### Example 3: Vascular Evaluation of Most Abundant Single Anthocyanins in Berry Extracts

The vascular properties of the single anthocyanins contained in Healthberry 865®: Delphinidin-3-rutinoside (D3-rut), Cyanidin-3-rutinoside (C3-rut), Delphinidin-3-glucoside (DP3-glu), Cyanidin-3-glucoside (C3-glu), Petunidin-3-glucoside (PT3-glu), Delphinidin-3-galactoside (DP3-gal), Peonidin-3-galactoside (PEO3-gal), Delphinidin-3-arabinoside (DP3-ara), Malvidin-3-galactoside (MAL3-gal), Malvidin-3-glucoside (MAL3-glu), Cyanidin-3-galactoside (C3-gal) and Cyanidin-3-arabinopyranoside (C3-arapy) were tested on mice mesenteric arteries.

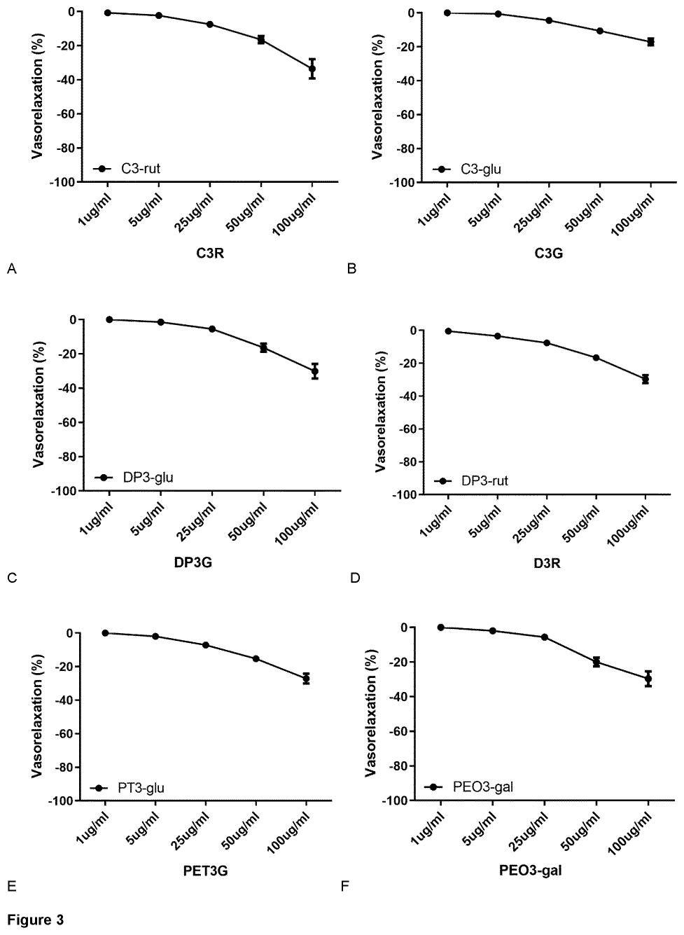

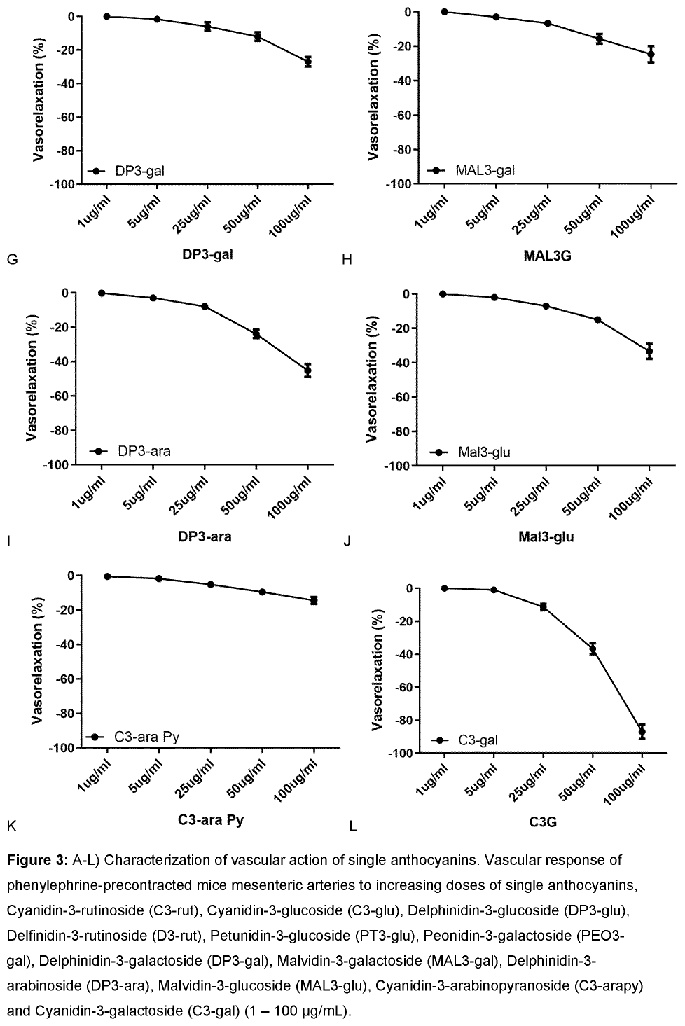

Interestingly, the evaluation of the possible direct vascular action of C3-rut, C3-glu, DP3-glu, PT3-glu, DP3-glu PEO3-gal, DP3-gal, MAL3-gal, DP3-ara and MAL3-glu revealed that none of the single anthocyanins was able to evoke a dose-dependent vasorelaxation comparable to that observed after Healthberry 865® administration (FIG. 3). A deeper analysis of dose-responses curves showed that DP3-gal and C3-rut reached better vasorelaxation compared to the other (about 32.5% and 36.5%). In contrast, only C3-gal and DP-3-Samb were able to reproduce the direct vasorelaxant effect of Healthberry 865®; in fact, the evoked vasorelaxant curve was very similar to that induced by Healthberry 865® (FIG. 3).

FIG. 3A-L) show characterization of vascular action of single anthocyanins. Vascular response of phenylephrine-precontracted mice mesenteric arteries to increasing doses of single anthocyanins, Cyanidin-3-rutinoside (C3-rut), Cyanidin-3-glucoside (C3-glu), Delphinidin glucoside (DP3-glu), Delfinidin-3-rutinoside (D3-rut), Petunidin-3-glucoside (PT3-glu), Peonidin galactoside (PEO3-gal), Delphinidin-3-galactoside (DP3-gal), Malvidin-3-galactoside (MAL3-gal), Delphinidin-3-arabinoside (DP3-ara), Malvidin-3-glucoside (MAL3-glu), Cyanidin arabinopyranoside (C3-arapy) and Cyanidin-3-galactoside (C3-gal) (1-100 μg/mL).

### Example 4: Vasorelaxant Action of Cyanidin-3-Galactoside and Delphinidin-3-Sambubioside is Mediated by AMPK/eNOS Signaling

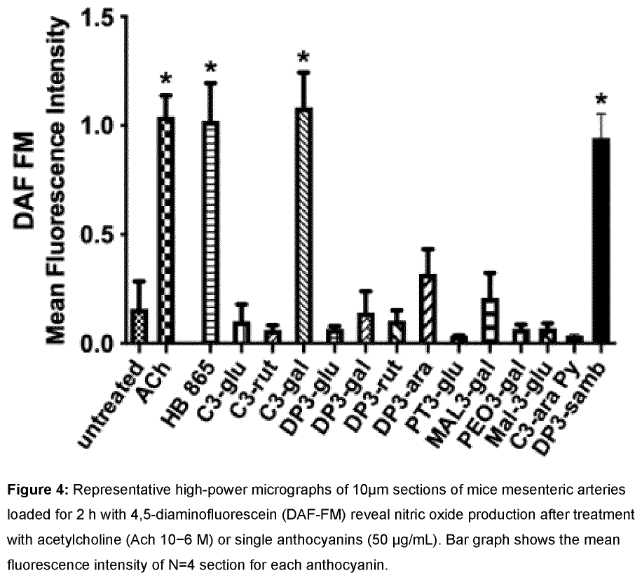

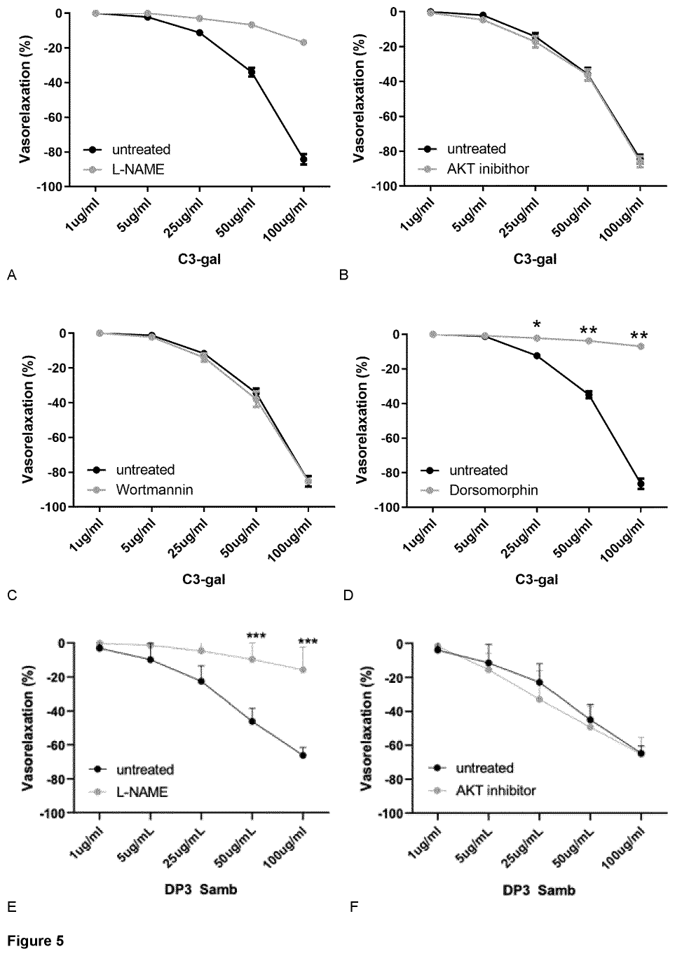

In order to evaluate the direct vascular action of the single anthocyanins on the modulation of nitric oxide synthase, which is the enzyme involved in Healthberry 865® evoked-vasorelaxation, the analysis and measurement of vessels-derived-nitric oxide after treatment of mesenteric arteries was performed with each anthocyanin. Interestingly, although vasorelaxant effects evoked by DP3-gal, C3-rut and DP3-ara were observed, only C3-gal and DP3-samb were able to evoke a significant increase of nitric oxide production from vessels, similarly to that observed after Healthberry 865® treatment (FIG. 4). Thus, the possible molecular mechanisms recruited by C3-gal and DP3-samb were analyzed. Of note, the inhibition of eNOS, by L-NAME, significantly reduced its vasorelaxant properties, thus confirming NO-dependent vasorelaxation. However, differently from what was observed in Healthberry 865® vascular action, the presence of Akt or PI3K inhibitor did not inhibit C3-gal vascular action, assuming a different upstream pathway. Thus, Dorsomorphin, an important selective inhibitor of AMPK was used. In this condition, C3-gal completely lost its vasorelaxant capability. Conversely, although Akt inhibitor did not influence DP3-Samb vascular action, it was inhibited both by the PI3K inhibitor, LY294002, and Dorsomorphin, thus demonstrating that Cyanidin-3-galactoside and Delphinidin-3-sambubioside exert vasorelaxant action using convergent pathways recapitulating the effect of Healthberry 865®. (FIG. 5).

FIG. 4 shows the representative high-power micrographs of 10 μm sections of mice mesenteric arteries loaded for 2 h with 4,5-diaminofluorescein (DAF-FM) reveal nitric oxide production after treatment with acetylcholine (Ach 10-6 M) or single anthocyanins (50 μg/mL). Bar graph shows the mean fluorescence intensity of N=4 section for each anthocyanin.

FIG. 5 shows vascular response of phenylephrine-precontracted mice mesenteric arteries to increasing doses of Cyanidin-3-galactoside (A) in presence of L-NAME, (B) in presence of Akt inhibitor, (C) in presence of PI3K inhibitor LY294002, (D) or in presence of Dorsomorphin, a selective AMPK inhibitor. Statistical analyses were performed using two-way ANOVA followed Bonferroni post-hoc test. *p<0.05; **p<0.01, ***p<0.001. E-H) Vascular response of phenylephrine-precontracted mice mesenteric arteries to increasing doses of Delphinidin-3-sambubioside (E) in presence of L-NAME, (F) in presence of Akt inhibitor, (G) in presence of PI3K inhibitor LY294002, (H) or in presence of Dorsomorphin.

### Example 5: The Antioxidant Vascular Action of Healthberry 865® is Due to the Combination of the Anthocyanins Contained

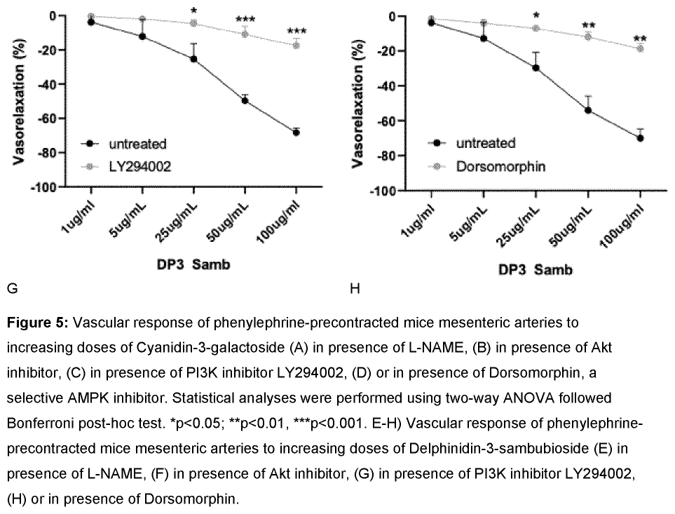

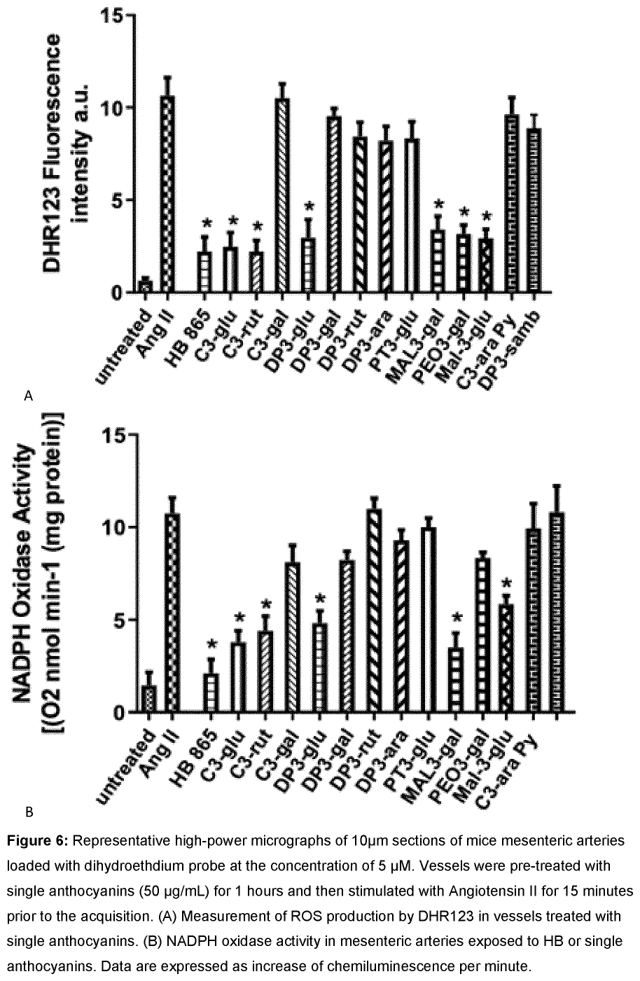

Previously few studies have reported an antioxidant activity of Healthberry 865® in human subjects (Karlsen et al. 2007). To investigate the capability of Healthberry 865® and the single anthocyanins contained on the modulation of oxidative stress, several methodological approaches were performed measuring both, total anti reactive oxygen species (ROS) capacity and their specific action on the modulation of the main machinery of ROS production, the activity of NADPH oxidase enzyme. The studies performed on mice mesenteric arteries revealed that Healthberry 865® owns an important anti-oxidative action, as showed by the significant reduction of Angiotensin II-induce ROS formation (FIG. 6). A deeper analysis, using single anthocyanins revealed that C3-glu, C3-rut, DP3-glu, MAL3-gal, PEO3-gal, Mal-3-glu are able to reproduce the antioxidant action of Healthberry 865®. Accordingly, the biochemical measurement of ROS generation by DHR1,2,3 probe confirm the results obtained with DHE (FIG. 6B).

Moreover, the analysis of NADPH oxidase (NOX) activity after stimulation with Angiotensin II, a gold-standard inducer of NOX activation was performed. The results showed that C3-glu, C3-rut, DP3-glu, MAL3-gal, PEO3-gal, MAL3-glu are able to reduce NOX activity. However, these single anthocyanins resulted in a smaller reduction than evoked by Healthberry 865® (FIG. 6B). In fact, C3-glu and MAL3-glu resulted to be the most powerful anthocyanins closer to the potent effect of Healthberry 865®.

FIG. 6 shows representative high-power micrographs of 10 μm sections of mice mesenteric arteries loaded with dihydroethdium probe at the concentration of 5 μM. Vessels were pre-treated with single anthocyanins (50 μg/mL) for 1 hours and then stimulated with Angiotensin II for 15 minutes prior to the acquisition. (A) Measurement of ROS production by DHR123 in vessels treated with single anthocyanins. (B) NADPH oxidase activity in mesenteric arteries exposed to HB or single anthocyanins. Data are expressed as increase of chemiluminescence per minute.

### Example 6: A Mix of Specific Anthocyanins Exert a Powerful Vasorelaxant and Antioxidative Action

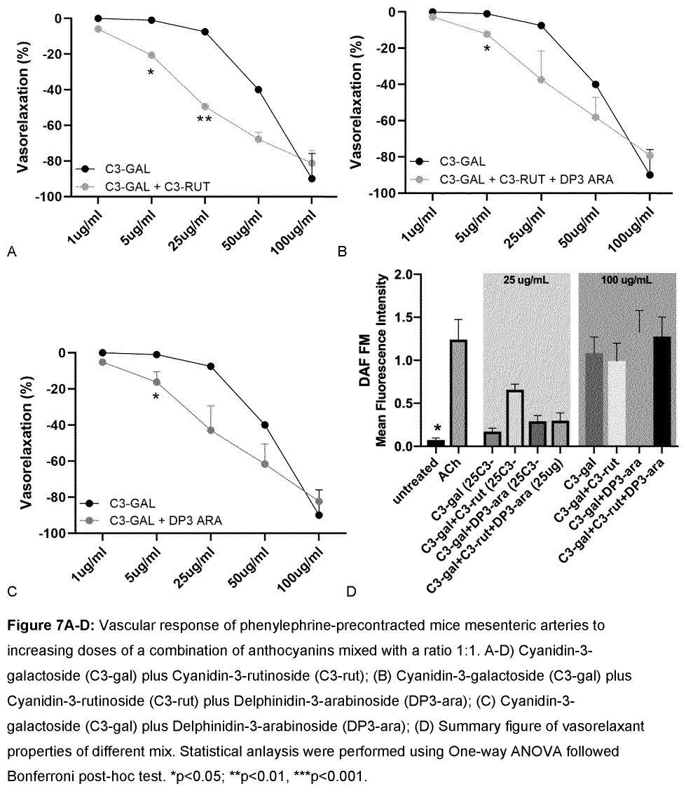

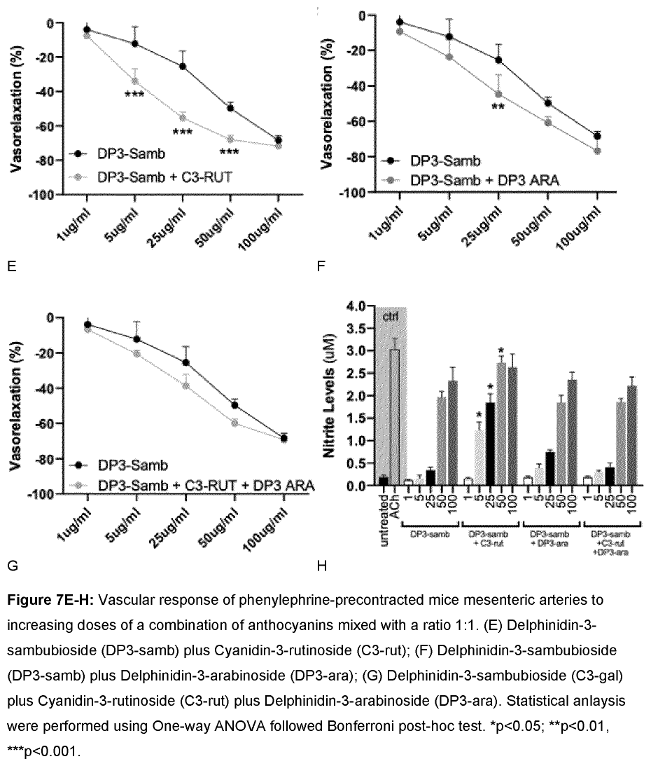

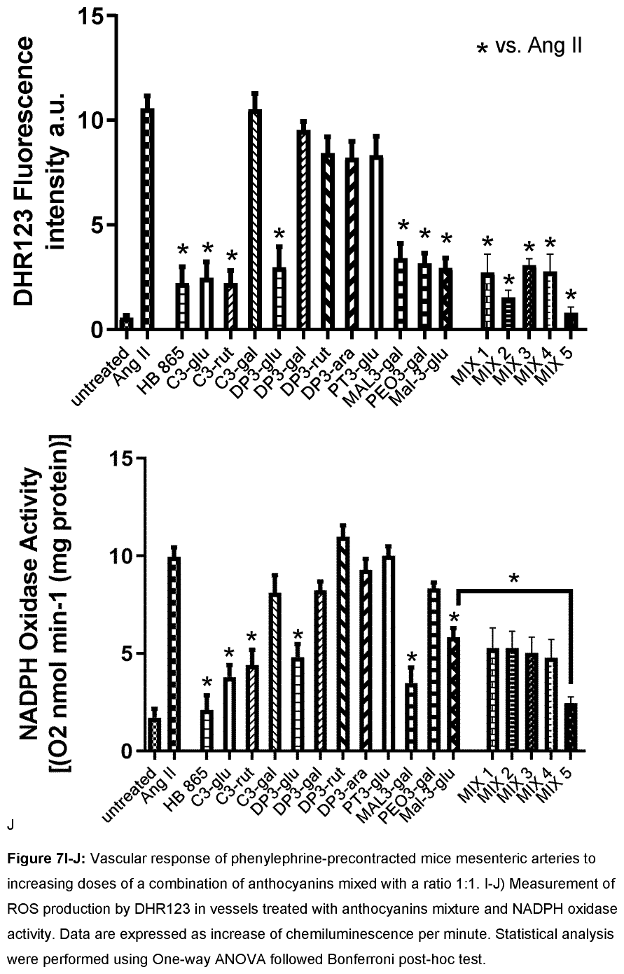

Based on the previous results, the possible effect on both vasorelaxation and antioxidative action of a mix of different Healthberry 865®-anthocyanins was investigated. To pursue this goal, C3-galactoside or DP3-samb, the most powerful vasorelaxant anthocyanins, was combined with C3-rut, DP3-ara, C3-rut, and DP3-ara/C3-rut in a triple combination, normalizing their relative concentration for each dose-response curve in order to obtain for a combination with two anthocyanins a ratio of 1/2:1/2 and for three 1/3:1/3:1/3. Surprisingly, in combination with C3-rut the best improvement of vasorelaxant curve has been observed, which although reaching the same maximal point of C3-gal alone, it showed a significant improvement of middle points (at 5 and 25 μg/mL) (FIG. 7A-D). Interestingly, the analysis of DAF-FM at the dosage of 25 μg/mL of each compound revealed a potentiation of nitric oxide release in presence of C3-gal plus C3-rut, in comparison to C3-gal alone.

FIG. 7A-D show vascular response of phenylephrine-precontracted mice mesenteric arteries to increasing doses of a combination of anthocyanins mixed with a ratio 1:1. A-D) Cyanidin-3-galactoside (C3-gal) plus Cyanidin-3-rutinoside (C3-rut); (B) Cyanidin-3-galactoside (C3-gal) plus Cyanidin-3-rutinoside (C3-rut) plus Delphinidin-3-arabinoside (DP3-ara); (C) Cyanidin galactoside (C3-gal) plus Delphinidin-3-arabinoside (DP3-ara); (D) Summary figure of vasorelaxant properties of different mix. (E) Representative high-power micrographs of 10 μm sections of mice mesenteric arteries loaded for 2 h with 4,5-diaminofluorescein (DAF-FM) reveal nitric oxide production after treatment with acetylcholine (Ach 10-6 M) or different anthocyanins mixed with a ratio 1/2:1/2 in presence of two anthocyanins of with a ratio 1/3:1/3:1/3 in presence of three compounds. Statistical analysis were performed using One-way ANOVA followed Bonferroni post-hoc test. *p<0.05; **p<0.01, ***p<0.001.

While, the combination of C3-gal/C3-rut/DP3-ara showed an increased release of nitric oxide only at 50 ug/mL. To characterize the efficacy of DP3-samb in combination we performed the same mix preparation used for C3-gal. Interestingly, DP3-samb in combination with C3-rut resulted more efficient to evoke vasorelaxation of mice mesenteric arteries, showing an important improvement from 5 to 50 ug/mL in comparison to that observed in combination with DP3-ara and DP3-ara/C3rut. Of note, nitrite measurement showed a significant increase at 5, 25 and 50 ug/mL thus confirming the most powerful efficacy of DP3-samb/C3-rut combination (FIG. 7E-H).

FIG. 7E-H show vascular response of phenylephrine-precontracted mice mesenteric arteries to increasing doses of a combination of anthocyanins mixed with a ratio 1:1. (E) Delphinidin-3-sambubioside (DP3-samb) plus Cyanidin-3-rutinoside (C3-rut); (F) Delphinidin-3-sambubioside (DP3-samb) plus Delphinidin-3-arabinoside (DP3-ara); (G) Delphinidin-3-sambubioside (C3-gal) plus Cyanidin-3-rutinoside (C3-rut) plus Delphinidin-3-arabinoside (DP3-ara). Statistical analysis were performed using One-way ANOVA followed Bonferroni post-hoc test. *p<0.05; **p<0.01, ***p<0.001.

To evaluate the role on oxidative stress, the action of further mixtures was analyzed: MIX 1: C3-glu+C3-gal; MIX 2: Mal3-glu+Mal3-gal; MIX 3: C3-glu+DP3-glu+Mal3-glu; MIX 4: Mal3-gal+PEO3-gal; MIX 5: C3-glu+DP3-glu+C3-rut+Mal3-glu+Mal3-gal+PEO3-gal. Interestingly, the measurement of both total ROS production and that of NADPH oxidase activity revealed highest efficacy of MIX 5 (FIGS. 7I-J).

FIGS. 7I-J show vascular response of phenylephrine-precontracted mice mesenteric arteries to increasing doses of a combination of anthocyanins mixed with a ratio 1:1. I-J) Measurement of ROS production by DHR123 in vessels treated with anthocyanins mixture and NADPH oxidase activity. Data are expressed as increase of chemiluminescence per minute. Statistical analysis were performed using One-way ANOVA followed Bonferroni post-hoc test.

### Example 7: Berry Extracts Reduce Oxidative Stress and Improve NO Bioavailability in Human Dysfunctional Vessels

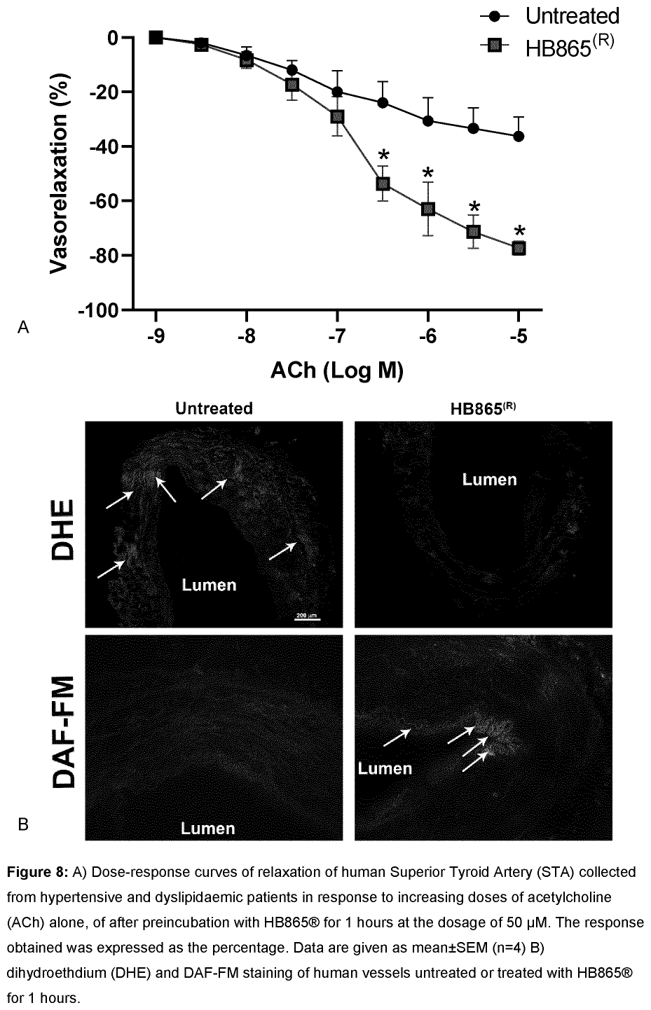

In order to translate the data obtained in animal models to human, the action of Healthberry 865® on human superior thyroid artery (STA) obtained from patients undergoing carotid revascularization surgery was assessed. At baseline, STA presented an important endothelial dysfunction, as showed by the altered acetylcholine-evoked vasorelaxation, while the muscular function resulted non compromised (FIG. 8). Of note, the treatment with 50 μg/mL of Healthberry 865® for 1 hour was able to significantly improve the altered endothelial vasorelaxation. The measurement of total oxidative stress revealed that after Healthberry 865® treatment there is a significant reduction of ROS in the vessels (FIG. 8). Surprisingly, after Healthberry 865® stimulation there is a significant increase of NO-production which reflects perfectly the improvement of vasorelaxant response.

FIG. 8 shows A) Dose-response curves of relaxation of human Superior Tyroid Artery (STA) collected from hypertensive and dyslipidaemic patients in response to increasing doses of acetylcholine (ACh) alone, of after preincubation with HB865® for 1 hours at the dosage of 50 μM. The response obtained was expressed as the percentage. Data are given as mean±SEM (n=4) B) dihydroethdium (DHE) and DAF-FM staining of human vessels untreated or treated with HB865® for 1 hours.

### Example 8: Mixture of Different Fruits for an Optimized Ratio of Anthocyanins with Vasorelaxant Activities

In order to achieve an optimal ratio of all anthocyanins, which have a strong antioxidative effect, literature values for the content of the single anthocyanins in specific fruits were compared. Since it is postulated that the beneficial anthocyanins shall be present in a nearly equimolar ratio, the fruits with the highest amounts of the respective anthocyanins were combined in different ratios to achieve balanced ratios of the anthocyanins cyanidin-3-galactoside, cyanidin-3-rutinoside, delphinidin-3-arabinoside.

The content of anthocyanins was analyzed in detail for black currant, black chokeberry bilberry, cowberry, elderberry, (Benvenuti et al., 2004; Kahkonen et al., 2003; Wu et al., 2004), strawberry, sweet cherry and sour cherry (Jakobek et al., 2007).

By mixing fruits with high amounts of the desired anthocyanins, the following contents of the specific anthocyanins were achieved:

TABLE 1
mixture of black currant, chokeberry, bilberry,
sweet cherry in the ratio of 1:1:1:1

| | | Total amount | |
|---|---|---|---|
| | Total amount | (weight-%/total | |
| | in mixture | anthocyanin | |
| Anthocyanin | (mg/100 g) | amount) | Ratio |
| cyanidin-3-galactoside | 1087 | 48 | 16 |
| cyanidin-3-rutinoside | 110 | 5 | 2 |
| delphinidin-3-arabinoside | 63 | 3 | 1 |
| others | 998 | 44 | 15 |
| Sum | 2259 | 100 | |

After mixing the desired berries in the ratio of 1:1:1:1, the specific anthocyanins are present in different amounts in the mixture, differing by a factor of up to 16.

By mixing fruits with high amounts of the desired anthocyanins in an optimized ratio, the following contents of the specific anthocyanins were achieved:

TABLE 2
mixture of black currant, chokeberry, bilberry,
sweet cherry in the ratio of 1:0.4:25:60

| | | Total amount | |
|---|---|---|---|
| | Total amount | (weight-%/total | |
| | in mixture | anthocyanin | |
| Anthocyanin | (mg/100 g) | amount) | Ratio |
| cyanidin-3-galactoside | 1621 | 11 | 1 |
| cyanidin-3-rutinoside | 1603 | 11 | 1 |
| delphinidin-3-arabinoside | 1575 | 11 | 1 |
| others | 9937 | 67 | 6 |
| Sum | 14736 | 100 | |

After mixing the desired berries in the ratio of 1:0.4:25:60, the specific anthocyanins are present in similar amounts in the mixture, differing by a factor of less than 2. This corresponds to the mixing ratio of anthocyanins from the previous experiments.

### REFERENCES

Alissa, E. M., and G. A. Ferns. 2012. ‘Functional foods and nutraceuticals in the primary prevention of cardiovascular diseases’, J Nutr Metab, 2012: 569486.
Asem, I. D., Imotomba, R. K., Mazumder, P. B., Laishram, J. M., 2015. ‘Anthocyanin content in the black scented rice (Chakhao):its impact on human health and plant defense’, Symbiosis (2015), 66(1), 47-54.
Bassenge, E., H. T. Schneider, and A. Daiber. 2005. ‘[Oxidative stress and cardiovascular diseases]’, Dtsch Med Wochenschr, 130: 2904-9.
Bell, D. R., and K. Gochenaur. 2006. ‘Direct vasoactive and vasoprotective properties of anthocyanin-rich extracts’, J Appl Physiol (1985), 100: 1164-70.
Benvenuti, S., Pellati, F., Melegari, M., Bertelli, D. (2006). ‘Polyphenols, Anthocyanins, Ascorbic Acid, and Radical Scavenging Activity of Rubus, Ribes, and Aronia’, Journal of Food Science, Vol, 69, Nr, 3, 2004
Carrizzo, A., M. Ambrosio, A. Damato, M. Madonna, M. Storto, L. Capocci, P. Campiglia, E. Sommella, V. Trimarco, F. Rozza, R. Izzo, A. A. Puca, and C. Vecchione. 2016. ‘Morus alba extract modulates blood pressure homeostasis through eNOS signaling’, Mol Nutr Food Res, 60: 2304-11.
Carrizzo, A., G. M. Conte, E. Sommella, A. Damato, M. Ambrosio, M. Sala, M. C. Scala, R. P. Aquino, M. De Lucia, M. Madonna, F. Sansone, C. Ostacolo, M. Capunzo, S. Migliarino, S. Sciarretta, G. Frati, P. Campiglia, and C. Vecchione. 2019. ‘Novel Potent Decameric Peptide of Spirulina platensis Reduces Blood Pressure Levels Through a PI3K/AKT/eNOS-Dependent Mechanism’, Hypertension, 73: 449-57.
Cassidy, A., M. Bertoia, S. Chiuve, A. Flint, J. Forman, and E. B. Rimm. 2016. ‘Habitual intake of anthocyanins and flavanones and risk of cardiovascular disease in men’, American Journal of Clinical Nutrition, 104: 587-94.
Ciocoiu, M., A. Miran, L. Mares, D. Tutunaru, C. Pohaci, M. Groza, M. Badescu: The effects of Sambucus nigra polyphenols on oxidative stress and metabolic disorders in experimental diabetes mellitus. In: Journal of physiology and biochemistry. Band 65, Nummer 3, September 2009, ISSN 1138-7548, S. 297-304
Curtis, P. J., P. A. Kroon, W. J. Hollands, R. Walls, G. Jenkins, C. D. Kay, and A. Cassidy. 2009. ‘Cardiovascular disease risk biomarkers and liver and kidney function are not altered in postmenopausal women after ingesting an elderberry extract rich in anthocyanins for 12 weeks’, J Nutr, 139: 2266-71.
de Pascual-Teresa, S., D. A. Moreno, and C. Garcia-Viguera. 2010. ‘Flavanols and anthocyanins in cardiovascular health: a review of current evidence’, Int J Mol Sci, 11: 1679-703.
Diczházi, I. and Kursinszki, L., 2014 ‘Anthocyanin Content and Composition in Winter Blue Barley Cultivars and Lines’, Cereal Chemistry, 91, 2, (195-200).
Escalante-Aburto, A., Ponce-Garcia, N., Ramirez-Wong, B., Torres-Chavez, P. I., de Dios Figueroa-Cardenas, J., Gutiérrez-Dorado, R., 2016. ‘Specific Anthocyanin Contents of Whole Blue Maize Second-Generation Snacks: An Evaluation Using Response Surface Methodology and Lime Cooking Extrusion’ Journal of Chemistry, Volume 2016
Faria, A., I. Fernandes, S. Norberto, N. Mateus, and C. Calhau. 2014. ‘Interplay between anthocyanins and gut microbiota’, J Agric Food Chem, 62: 6898-902.
Hassellund, S. S., A. Flaa, S. E. Kjeldsen, I. Seljeflot, A. Karlsen, I. Erlund, and M. Rostrup. 2013. ‘Effects of anthocyanins on cardiovascular risk factors and inflammation in pre-hypertensive men: a double-blind randomized placebo-controlled crossover study’, Journal of Human Hypertension, 27: 100-06.
Higashi, Y., K. Noma, M. Yoshizumi, and Y. Kihara. 2009. ‘Endothelial function and oxidative stress in cardiovascular diseases’, Circ J, 73: 411-8.
Hosseinian FS1, Beta T. 2007 ‘ Saskatoon and wild blueberries have higher anthocyanin contents than other Manitoba berries’, Journal of Agricultural and Food Chemistry, 55(26), 10832-10838.
Hou, D. X. 2003. ‘Potential mechanisms of cancer chemoprevention by anthocyanins’, Curr Mol Med, 3: 149-59.
Huxley, R. R., and V. Perkovic. 2014. ‘The modifiable burden of worldwide mortality from cardiovascular diseases’, Lancet Diabetes Endocrinol, 2: 604-6.
Jakobek L., Seruga, M., Novak, I, Medividovic-Kasonavic, M. 2007. ‘Flavonols, Phenolic Acids and Antioxidant Activity of Some Red Fruits’, Deutsche Lebensmittel-Rundschau, 103, Jahrgang, Heft 2, 2007
Kähkönen, M. P., Heinämäki, J., Ollilainen, V. and Heinonen, M 2003. ‘Berry anthocyanins: isolation, identification and antioxidant activities’, J Sci Food Agric 83:1403-1411
Karlsen, A., L. Retterstol, P. Laake, I. Paur, S. K. Bohn, L. Sandvik, and R. Blomhoff. 2007. ‘Anthocyanins inhibit nuclear factor-kappaB activation in monocytes and reduce plasma concentrations of pro-inflammatory mediators in healthy adults’, J Nutr, 137: 1951-4.
Khoo, H. E., A. Azlan, S. T. Tang, and S. M. Lim. 2017. ‘Anthocyanidins and anthocyanins: colored pigments as food, pharmaceutical ingredients, and the potential health benefits’, Food & Nutrition Research, 61: 1-21.
Li, C. Y., L. X. Wang, S. S. Dong, Y. Hong, X. H. Zhou, W. W. Zheng, and C. Zheng. 2018. ‘Phlorizin Exerts Direct Protective Effects on Palmitic Acid (PA)-Induced Endothelial Dysfunction by Activating the PI3K/AKT/eNOS Signaling Pathway and Increasing the Levels of Nitric Oxide (NO)’, Med Sci Monit Basic Res, 24: 1-9.
Lind, L., L. Berglund, A. Larsson, and J. Sundstrom. 2011. ‘Endothelial function in resistance and conduit arteries and 5-year risk of cardiovascular disease’, Circulation, 123: 1545-51.
Mishra, R., and Monica. 2019. ‘Determinants of cardiovascular disease and sequential decision-making for treatment among women: A Heckman's approach’, Ssm-Population Health, 7.
Ojeda, D., Jiménez-Ferrer, E., Zamilpa, A., Herrera-Arellano, A., Tortoriello, J., Alvarez, L. 2009. ‘Inhibition of angiotensin convertin enzyme (ACE) activity by the anthocyanins delphinidin- and cyanidin-3-O-sambubiosides from Hibiscus sabdariffa.’, J Ethnopharmacol. 2010 Jan. 8; 127(1):7-10.
Puca, A. A., A. Carrizzo, F. Villa, A. Ferrario, M. Casaburo, A. Maciag, and C. Vecchione. 2013. ‘Vascular ageing: the role of oxidative stress’, Int J Biochem Cell Biol, 45: 556-9.
Qin, Y., M. Xia, J. Ma, Y. Hao, J. Liu, H. Mou, L. Cao, and W. Ling. 2009. ‘Anthocyanin supplementation improves serum LDL- and HDL-cholesterol concentrations associated with the inhibition of cholesteryl ester transfer protein in dyslipidemic subjects’, American Journal of Clinical Nutrition, 90: 485-92.
Schiattarella, G. G., A. Carrizzo, F. Ilardi, A. Damato, M. Ambrosio, M. Madonna, V. Trimarco, M. Marino, E. De Angelis, S. Settembrini, C. Perrino, B. Trimarco, G. Esposito, and C. Vecchione. 2018. ‘Rac1 Modulates Endothelial Function and Platelet Aggregation in Diabetes Mellitus’, J Am Heart Assoc, 7.
Schmitt, J., and A. Ferro. 2013. ‘Nutraceuticals: is there good science behind the hype?’, British Journal of Clinical Pharmacology, 75: 585-87.
Seo et al., 2011 ‘Relationship of radical scavenging activities and anthocyanin contents in the 12 colored rice varieties in Korea’, Journal of the Korean Society for Applied Biological Chemistry, 54(5), 693-699.
Takeoka, G. R., Dao, L., Harden, L., Pantoja, A., Kuhl, J. C. 2013. ‘Antioxidant activity, phenolic and anthocyanin contents of various rhubarb (Rheum spp.) varieties’, International Journal of Food Science and Technology, 48(1), 172-178.
Tsuda, T., F. Horio, K. Uchida, H. Aoki, and T. Osawa. 2003. ‘Dietary cyanidin 3-O-beta-D-glucoside-rich purple corn color prevents obesity and ameliorates hyperglycemia in mice’, J Nutr, 133: 2125-30.
Vecchione, C., E. Patrucco, G. Marino, L. Barberis, R. Poulet, A. Aretini, A. Maffei, M. T. Gentile, M. Storto, O. Azzolino, M. Brancaccio, G. L. Colussi, U. Bettarini, F. Altruda, L. Silengo, G. Tarone, M. P. Wymann, E. Hirsch, and G. Lembo. 2005. ‘Protection from angiotensin II-mediated vasculotoxic and hypertensive response in mice lacking PI3Kgamma’, J Exp Med, 201: 1217-28.
Wallace, T. C. 2011a. ‘Anthocyanins in Cardiovascular Disease’, Advances in Nutrition, 2: 1-7. - - - . 2011b. ‘Anthocyanins in cardiovascular disease’, Advances in Nutrition, 2: 1-7.
Wallace, T. C., M. Slavin, and C. L. Frankenfeld. 2016. ‘Systematic Review of Anthocyanins and Markers of Cardiovascular Disease’, Nutrients, 8.
Wu X, Gu L, Prior R L, McKay S. 2004 ‘Characterization of anthocyanins and proanthocyanidins in some cultivars of Ribes, Aronia, and Sambucus and their antioxidant capacity’, J, Agric, Food Chem, 52, 7846-7856.

1. A composition suitable for use in vascular relaxation, the composition comprising:
cyanidin-3-galactoside having a formula: 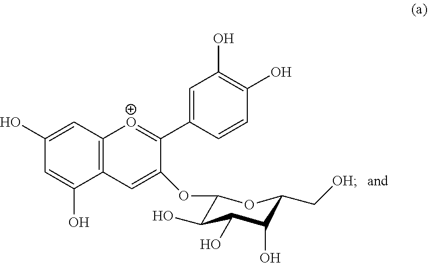
(b) cyanidin-3-rutinoside (b-i), delphinidin-3-arabinoside (b-ii), and delphinidin-3-sambubioside (b-iii).

2. The composition of claim 1, further comprising:
a fruit or fruit extract.

3. The composition of claim 1, comprising:
bilberry, sweet cherry, and cowberry/lingonberry, optionally as extract(s).

4. The composition of claim 1, comprising:
an extract of black currant and bilberry.

5. The composition of claim 4, wherein the black currant is from Ribes nigrum and/or the bilberry is from Vaccinium myrtillus.

6. The composition of claim 1, comprising anthocyanins at a concentration of at least 25 wt. %.

7. The composition of claim 2, wherein the extract is present and is an alcoholic extract, preferably a methanol extract.

8. The composition of claim 2, wherein the extract is present and is prepared by a process comprising:
extracting a black currant and/or bilberry, to obtain an extract;
purifying the extract via chromatography, to obtain a purified extract;
mixing the purified extract(s) with water, to obtain a mixture; and
spray-drying the mixture.

9. A method for preventing or treating a cardiovascular disease or disorder, the method comprising:
providing a subject in need thereof with the composition of claim 1, thereby prevent or treating the disease or disorder.

10. A composition, comprising:
black currant, (black) chokeberry, bilberry, and sweet cherry, optionally as extract(s),
wherein the composition comprises anthocyanins and the anthocyanins are present in the composition in a concentration of at least 5 μg/mL.

11. The composition of claim 10, further comprising:
cowberry, lingonberry, hibiscus, or elderberry, optionally as extract(s).

12. The composition of claim 10, comprising:
the black currant, (black) chokeberry, bilberry, and sweet cherry or extract in a weight ratio in a range of from 0.5 to 5:0.25 to 2.5:15 to 35:50 to 70.

13. The composition of claim 12, comprising:
the black currant, (black) chokeberry, bilberry, and sweet cherry in a weight ratio of around 1:0.4:25:60.

14. The composition of claim 1, further comprising:
bilberry, black currant, and/or black chokeberry, optionally as extract(s).

15. The composition of claim 1, further comprising:
cowberry/lingonberry and bilberry, optionally as extract(s).

16. The composition of claim 1, further comprising:
black scented rice; and
sweet cherry or black currant or red currant,
optionally as extract(s).

17. The composition of claim 4, comprising the extract from the black currant and the bilberry in a weight ratio in a range of from 0.5:1 to 1:0.5.

18. The composition of claim 2, wherein the extract is present and is a methanol extract.

19. A method for preventing or treating a atherosclerosis, hypertension, stroke, diabetes-related cardiovascular disfunctions, ischemia/reperfusion injury, hypercholesterolemia, coronary artery disease, or chronic obstructive pulmonary disease, the method comprising:
providing a subject in need thereof with the composition of claim 1, thereby preventing or treating the atherosclerosis, hypertension, stroke, diabetes-related cardiovascular disfunctions, ischemia/reperfusion injury, hypercholesterolemia, coronary artery disease, or chronic obstructive pulmonary disease.
